(19) United States
(12) Patent Application Publication
(10) Pub. No.: US 2022/0005595 A1
(43) Pub. Date: Jan. 6, 2022

(54) SYSTEM AND METHOD FOR VIRTUAL ONLINE ASSESSMENT OF MEDICAL TRAINING AND COMPETENCY

(71) Applicant: Abdul Karim Qayumi, West Vancouver (CA)

(72) Inventor: Abdul Karim Qayumi, West Vancouver (CA)

(21) Appl. No.: 17/367,515

(22) Filed: Jul. 5, 2021

Related U.S. Application Data
(60) Provisional application No. 63/048,012, filed on Jul. 3, 2020.

(51) Int. Cl. G16H 40/20; G16H 50/20; G06N 20/00; G09B 5/00

(52) U.S. Cl. CPC G16H 40/20; G16H 50/20; G06Q 10/0639; G09B 5/00; G06N 20/00

(57) ABSTRACT

There is disclosed a system and method for providing an assessment of medical competencies. In an embodiment, the method comprises: providing a virtual interactive environment for access by an expert and by a student; providing an artificial intelligence machine learning engine for generating random scenarios and interactions for testing competencies; and based on the student's response to the random scenarios and interactions, performing a machine assessment of the student's competency. In another embodiment, the method further comprises providing an expert assessment by the expert of the student's competency. In another embodiment, the method further comprises combining the machine assessment and the expert assessment to calculate an overall score.

### CROSS-REFERENCE TO RELATED APPLICATIONS

This application claims the benefit of U.S. Provisional Patent Application No. 63/048,012 filed on Jul. 3, 2020, which is incorporated by reference herein in its entirety.

### FIELD OF THE INVENTION

The present disclosure relates generally to medical training and competency assessment.

### BACKGROUND

Undergraduate and post graduate medical education is moving towards competency-based education (CBE). Assessment of competency-based education create certain challenges for the medical education system. The main challenges include substantial increase in manpower (faculty) and cost of medical education. For example, medical students' knowledge and readiness for practice, after graduation, is assessed mainly by a multiple-choice question (MCQ) examination. Recently (2013), the Association of Faculties of Medicine of Canada (AFMC) and in 2014 the Association of American Medical Colleges (AAMC) came up with a new assessment to determine if a medical graduate is ready for practice. The assessment is called Entrustable Professional Activities (EPAs). EPAs are a set of practical competencies (12 in Canada and 13 in the US) required for medical graduate to be able to perform, and should be assessed for to make sure they are ready for practice. This means that every graduate student should be followed by qualified faculty for at least a week or so for assessment of all competencies required. This can take place in a clinical environment with patients or in a simulation facility. Therefore, the cost of this assessment will be much higher in comparison to the traditional MCQ exam where hundreds of students can sit in a facility to complete their examination.

In recent years, many undergraduate and post graduate education programs are changing their curriculum into competency-based education. This means that most assessments in undergraduate and post graduate education will be changed to competency-based assessment. It is proven that CBE will significantly improve the quality of medical education, however, it would also impose enormous challenges to the medical education system.

This revolutionary change in medical education—from theoretical to competency-based—create significant challenges, including the following: (1) Competency-based assessment require much more manpower i.e. faculty to assess every student; (2) It requires space equipment and staff to operate the assessment environment if it is a hospital, clinic, simulation center or others; (3) It is, therefore, substantially increase the cost of medical education. (4) It requires a convenient time for all parties to be at the same place (students, faculty, staff, patients, technicians and others); (5) It cannot be 100% standard for all students having their assessment in verity of places, (universities and hospitals and others) assessors. The quality of assessment will be depending on the quality of instructors, examiners, culture of the place and others; and (6) Cannot be repeatedly performed, therefore, students cannot practice for the assessment.

Currently, assessing the practical aspects of medical knowledge is performed using the following three methods: (1) Real patients—this has legal ethical consequences and it is costly; (2) Simulated patients—these are artistes trained to be a patient. It is also costly and bound to time and space; (3) High fidelity simulators—these are mannequins that can act like a patient. Also costly to buy and maintain it and has limited ability to simulate many features and physiological parameters or verity of conditions. Each of these methods has significant drawbacks given their cost and limited availability.

Therefore, what is needed is a way to perform competency-based assessments in a more practical and efficient manner, which addresses at least some of the above described challenges.

### SUMMARY

The present disclosure relates generally to a system and method for virtual online assessment of medical training and competency.

There is disclosed a system and method for providing an assessment of medical competencies. In an embodiment, the method comprises: providing a virtual interactive environment for access by an expert and by a student; providing an artificial intelligence machine learning engine for generating random scenarios and interactions for testing competencies; and based on the student's response to the random scenarios and interactions, performing a machine assessment of the student's competency. In another embodiment, the method further comprises providing an expert assessment by the expert of the student's competency. In another embodiment, the method further comprises combining the machine assessment and the expert assessment to calculate an overall score.

In an aspect, there is disclosed a method of assessment of medical competencies, comprising: providing a virtual interactive environment for access by an expert and by a student; providing an artificial intelligence machine learning engine for generating random scenarios and interactions for testing competencies; and based on the student's response to the random scenarios and interactions, performing a machine assessment of the student's competency.

In an embodiment, the method further comprises providing an expert assessment by the expert of the student's competency.

In another embodiment, the method further comprises combining the machine assessment and the expert assessment to calculate an overall score.

In another embodiment, the method further comprises comparing the machine assessment to the expert assessment to perform machine learning, and to update an algorithm to be used in a subsequent machine assessment.

In another embodiment, the method further comprises providing a student self-assessment of the student's competency during training sessions.

In another aspect, there is provided a system for performing an assessment of medical competencies, comprising: a virtual interactive environment for access by an expert and by a student; an artificial intelligence machine learning engine for generating random scenarios and interactions for testing competencies; and a machine assessment module for performing a machine assessment of the student's competency-based on the student's response to the random scenarios and interactions.

In an embodiment, the system further comprises an expert assessment module for enabling an expert assessment of the student's competency.

In another embodiment, the system is configured to calculate an overall score by combining the machine assessment and the expert assessment.

In another embodiment, the system is further configured to compare the machine assessment to the expert assessment to perform machine learning, and to update an algorithm to be used in a subsequent machine assessment.

In another embodiment, the system further comprises a student self-assessment module configured to allow the student to perform a self-assessment of the student's competency during training sessions.

In another embodiment, the system and method utilizes animated objects in the virtual environment in any form or dimension for learning or assessment of medical competencies.

In another embodiment, the system and method utilizes still art and pictures, audio, video, or any other type of media.

In another embodiment, the system and method provides digital audio for learning or assessment of medical competencies.

In another embodiment, the system and method utilizes any combination of audio, video, animation, Avatars and other digital materials for the purpose of building an assessment platform foe evaluation and assessment of medical competencies.

Advantageously, the system and method is substantially less manpower driven, it is very cost-effective, and it does not require space, equipment, and staff to operate. Furthermore, the system and method provides the environment for students to repeat it and practice as many times as needed, and be tailored to each student.

As the system is accessible online, it is not bound to time and space, and can be reached at any time from any place.

As well, the system is capable of assessing all kinds of competencies, such as professionalism, advocacy communication, documentation and other competencies in the continuum of care.

In this respect, before explaining at least one embodiment of the invention in detail, it is to be understood that the invention is not limited in its application to the details of construction and to the arrangements of the components set forth in the following description or the examples provided therein, or illustrated in the drawings. Therefore, it will be appreciated that a number of variants and modifications can be made without departing from the teachings of the disclosure as a whole. Therefore, the present system, method and apparatus is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

The present system and method will be better understood, and objects of the invention will become apparent, when consideration is given to the following detailed description thereof. Such description makes reference to the annexed drawings, wherein:

FIG. 1 shows a schematic overview of the present system and method in accordance with an embodiment.

FIG. 2 shows a schematic diagram of system features for assessing history taking competencies in accordance with an embodiment.

FIG. 3 shows a schematic diagram of system features for assessing physical examination competencies in accordance with an embodiment.

FIG. 4 shows a schematic diagram of system features for assessing investigation competencies in accordance with an embodiment.

FIG. 5 shows a schematic diagram of system features for assessing management plane competencies in accordance with an embodiment.

FIG. 6 shows a schematic diagram of system features for assessing patient transfer competencies in accordance with an embodiment.

FIG. 7 shows a schematic diagram of system features for assessing ancillary care competencies in accordance with an embodiment.

FIG. 8 shows a schematic diagram of system features for assessing competencies for delivering difficult news in accordance with an embodiment.

FIG. 9 shows a schematic diagram of system features for assessing quality improvement competencies in accordance with an embodiment.

FIG. 10 shows a schematic diagram of system features for assessing preventative competencies in accordance with an embodiment.

FIG. 11 shows a schematic block diagram of a computer system which may provide an operating environment for various embodiments.

In the drawings, embodiments are illustrated by way of example. It is to be expressly understood that the description and drawings are only for the purpose of illustration and as an aid to understanding, and are not intended as describing the accurate performance and behavior of the embodiments and a definition of the limits of the invention.

### DETAILED DESCRIPTION

As noted above, the present disclosure relates to a system and method for virtual online assessment of medical training and competency.

In an aspect, there is provided a system for assessing the medical training and competency of individuals, in particular medical students.

The present system is built on a technologically advanced digital platform to support assessment and/or practice for assessment of competency-based medical education.

In contrast to prior methods of assessment, the present system provides a cost effective, easily accessible online platform which provides a virtual assessment environment for assessing a continuum of care.

In an embodiment, the physical structure of this tool/system utilizes a “CyberPatient” (CP) platform which is accessed by a subject being assessed, as well as those assessing the subject to determine how the subject performs. In a preferred embodiment, the system supports avatars including patients, doctors, nurses, patient families, care takers and others. These roles are programed to respond to the outside triggers caused by the subject student.

In another embodiment, the system is designed to simulate the way doctors have interaction with patients in a real hospital room, clinic, emergency room or other medical environments. However, all of these environments are created in cyberspace, and the avatars programmed in the environment act like real patients. This will give the opportunity for students to practice medicine without harming the patient and increasing the cost of the healthcare system.

In another embodiment, the tool/system is equipped with an artificial intelligence (AI) engine that is based on algorithms, pattern recognition and machine learning, herein called an “Intelligent Tutoring System” or ITS. This ITS collects data generated in an assessment session, and provides feedback to the student in real-time. Therefore, the student will have an immediate feedback (self-assessment) when the system is operation. In this self-assessment, additional data such as audio/visual data and documentation that are important in medical practice is also collected during the time when the student is interacting with the CP. In the end of the case, in addition to the machine data templates of correct communication, documentation will be provided to the students so the student can have the opportunity to compare their communication and documentation with a “gold-standard” provided as a reference point in the system.

In an embodiment, the system also has a third-party assessment mode where collected data will be sent to a specialist in the field to assess the student. These assessment modes have specific purposes. A first mode is designed for students to practice their competency assessment, and a second mode is designed exclusively for an online assessment of competency-based education.

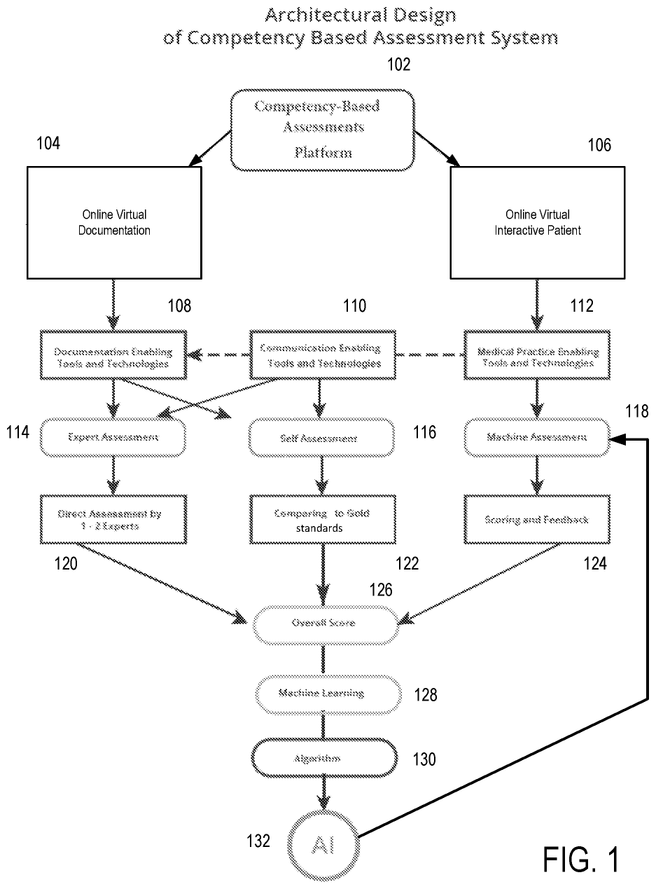

In an illustrative embodiment, the system comprises one or more of the following features:
1) Algorithm in the discerption of the competencies,
2) Recognition and discerption of the behavioral pattern for specific competencies
3) Identification of the state-of-the-art technology including but not limited to:
   a. Audio
   b. Video
   c. Interactive online smart forms
   d. Interactive online smart documents
   e. Virtual presentation
   f. others
4) Design interactivity in the system for each competency at a point of care or for the entire continuum of the care.
5) Design a strong database system for data collection, distribution and storage.
6) Instant data analysis and feedback engine.
7) Artificial intelligence capabilities through Machine learning, algorithms, pattern recognition.
Structure of the system/method is depicted in FIG. 1 (Please see attached pdf document).

In another embodiment, the structure of this competency-based assessment system includes an online platform capable of providing tools and technologies for assessment of medical competencies in virtual environment. The platform consists of the following components:
1. Information about Specific medical competencies and how to assess them.
2. Online virtual patient in the cyberspace capable of simulation a real patient.
3. Medical practice enabling tools and technologies capable of simulating the competencies and their assessments in a virtual space
4. Virtual online tools and technologies capable of simulating patient chart for documentation of events and processes in the continuum of care.
5. Communication enabling tools and technologies facilitating, recording, replaying, archiving and analyzing conversations between student, patient, patient family/caretaker, the team and others.
6. Enabling data basis for questions asked form the patient, physical examination, lab tests, imaging, medication, fluids and others
7. Machine assessment capabilities (Intelligent Tutoring System) on logic and interactivity of the student's interaction with virtual patient and/or competency-based learning tools and technologies providing scoring and feedback to the student for self-directed learning.
8. Self-assessment capabilities capable of comparing their performance with the gold standard that supports reflection and self-directed learning.
9. Expert assessment provides the opportunity to record and display the unbiased opinion of external experts on the performance of the students.
10. Artificial Intelligence (AI) capabilities by using:
   a. Score gathered from all three assessment
   b. Algorithm provided to the system
   c. Machine learning capabilities

In operation, the system performs an assessment utilizing an AI algorithm which is a learning algorithm, and which assessment is further improved with each iteration. For training purposes, one or two subject-matter experts may perform an assessment in parallel with the machine assessment to validate the machine assessment. The student may also perform a self-assessment as to how they believe they have performed, and the combination of all three assessments is used to calculate an overall score.

Advantageously, the present system and method provides a virtual online environment for assessing competencies in medical education which is efficient and cost effective, as the resources required to perform the assessment are just a fraction of what they would be if done traditionally.

In addition, the machine assessment performed by the system improves with each iteration, as one or two subject-matter experts may perform an assessment in parallel to validate the machine assessment.

With reference to the drawings, FIG. 1 shows a schematic overview of the present system and method in accordance with an embodiment. A competency-based assessment platform 102 comprises online virtual documentation 104 and provides an online virtual interactive patient 106. Documentation enabling tools and technologies 108 and communication enabling technologies 110 enable an expert assessment 114 and a self-assessment 116 by the student. A machine assessment 118 performed by the system is also enabled.

In an embodiment, a direct assessment 120 performed by one or two experts is combined with a self-assessment comparison to a “gold standard” 122, and with automated scoring and feedback 124 performed by the system to calculate an overall score 126. The combined score is provided as an input to a machine learning module 128 to generate an improved algorithm 130 for the system's AI 132. The system's AI is utilized by machine assessment module 118 for a subsequent assessment, and this iterative process allows the system to continually improve its assessment performance over time.

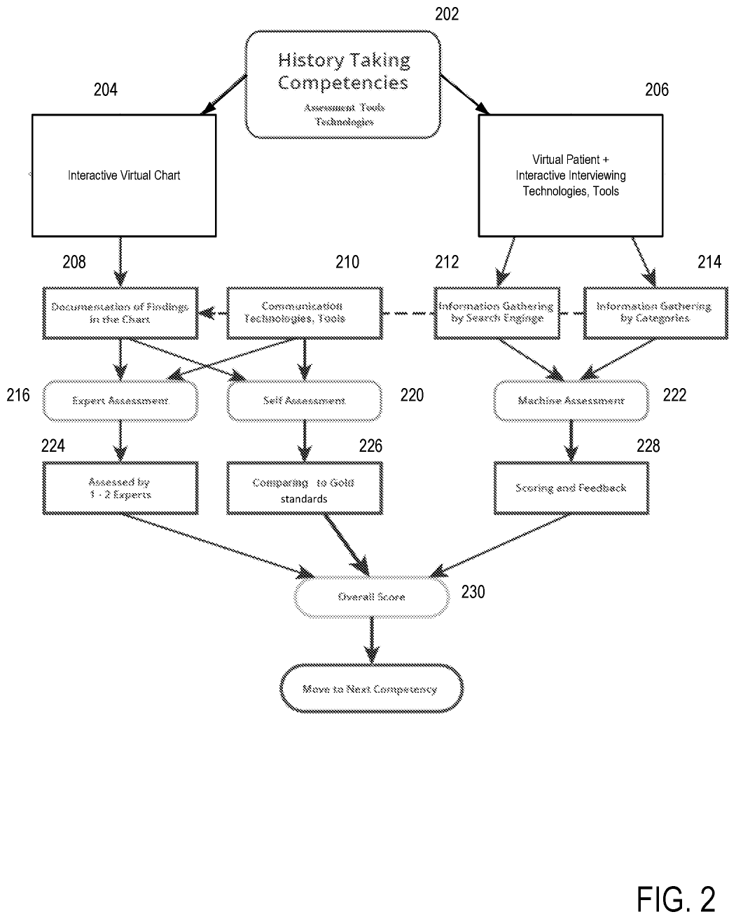

FIG. 2 shows a schematic diagram of system features for assessing history taking competencies in accordance with an embodiment. This module for history taking competencies 202 comprises an interactive virtual chart 204 and a virtual patient interactive interviewing technologies and tools 206. Documentation of findings in the chart 208 and communication technologies and tools 210 facilitate an expert assessment 216 and a self-assessment 220. The system may facilitate assessment by one or two experts 224 and a self-assessment as compared to a gold standard 226.

The system also includes information gathering by search engines 212 and information gathering by categories 214. These three assessments 216, 220, 222 are combined into an overall score 230.

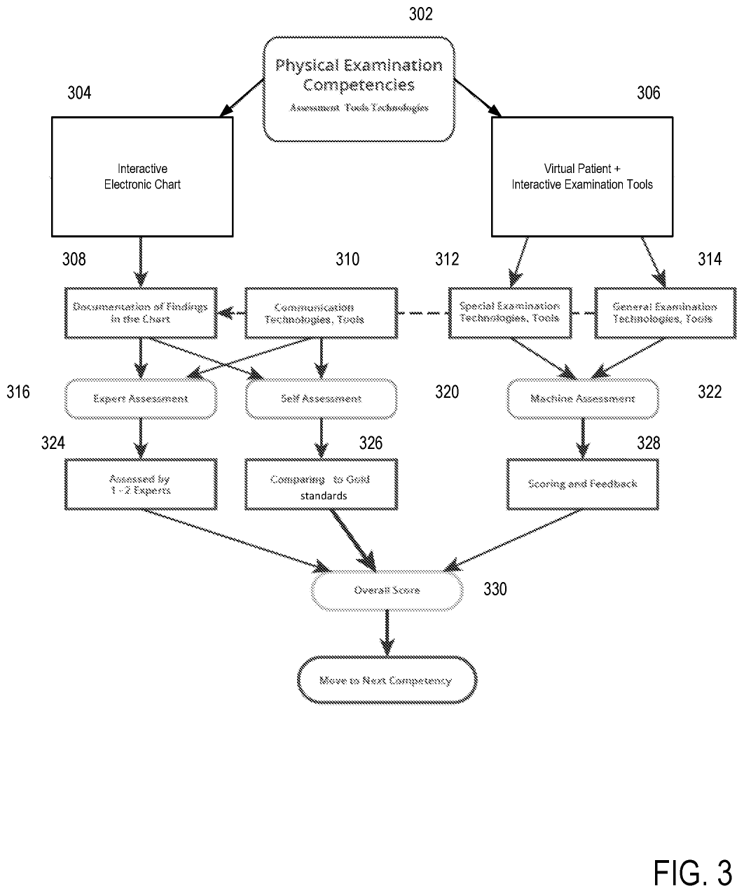

FIG. 3 shows a schematic diagram of system features for assessing physical examination competencies in accordance with an embodiment. This module for history taking competencies 302 comprises an interactive electronic chart 304 and a virtual patient interactive examination tool 306. Documentation of findings in the chart 308 and communication technologies and tools 310 facilitate an expert assessment 316 and a self-assessment to 320. The system may facilitate assessment by one or two experts 324 and a self-assessment as compared to a gold standard 326. The system also includes special examination technologies and tools 312, and general examination technologies 314. Assessment by one or two experts 324, self-assessment by student compared to a “gold standard” 326, and scoring and feedback 328 provided by the machine assessment may all be combined into an overall score 330.

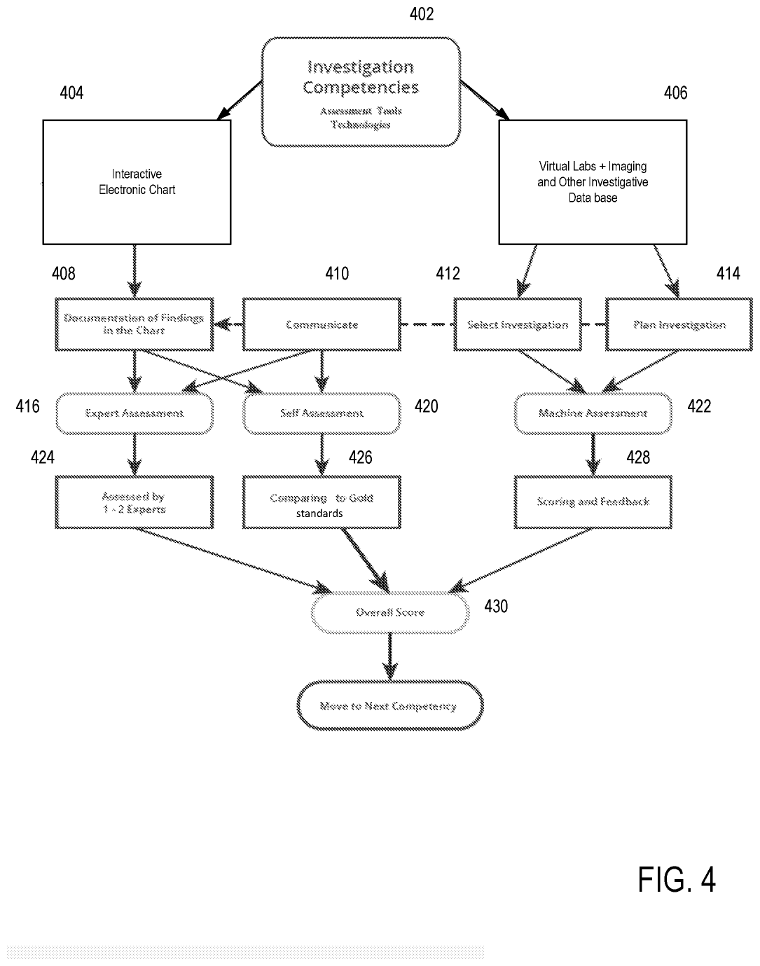

FIG. 4 shows a schematic diagram of system features for assessing investigation competencies in accordance with an embodiment. As shown, the system 402 comprises an interactive electronic chart 404 and a virtual lab 406. Documentation of findings in the chart 408 and communication technologies and tools 410 facilitate an expert assessment 416 and a self-assessment to 420. The system may facilitate assessment by one or two experts to 424 and a self-assessment as compared to a gold standard 426. The system also includes special examination technologies and tools 412, and general examination technologies 414. Assessment by one or two experts 424, self-assessment by student compared to a “gold standard” 426, and scoring and feedback 428 provided by the machine assessment may all be combined into an overall score 430.

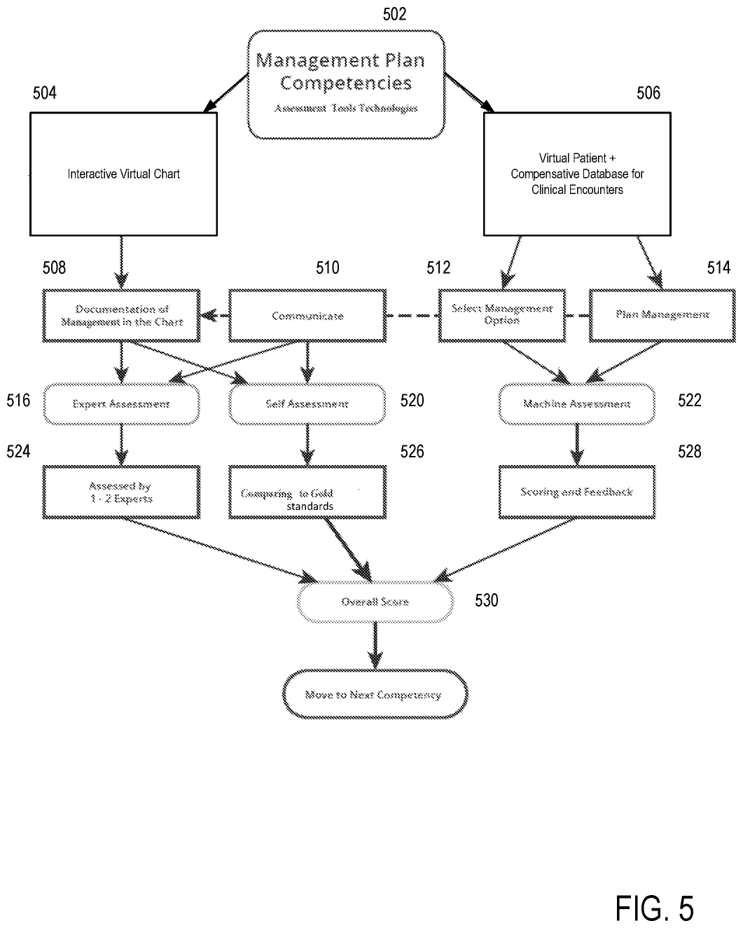

FIG. 5 shows a schematic diagram of system features for assessing management plane competencies in accordance with an embodiment. As shown, the system 502 comprises an interactive electronic chart 502 and a virtual patient and compensative data 506. Documentation of findings in the chart 508 and communication technologies and tools 510 facilitate an expert assessment 516 and a self-assessment to 520. The system may facilitate assessment by one or two experts to 524 and a self-assessment as compared to a gold standard 526. The system also includes special examination technologies and tools 512, and general examination technologies 514. Assessment by one or two experts 524, self-assessment by student compared to a “gold standard” 526, and scoring and feedback 528 provided by the machine assessment may all be combined into an overall score 530.

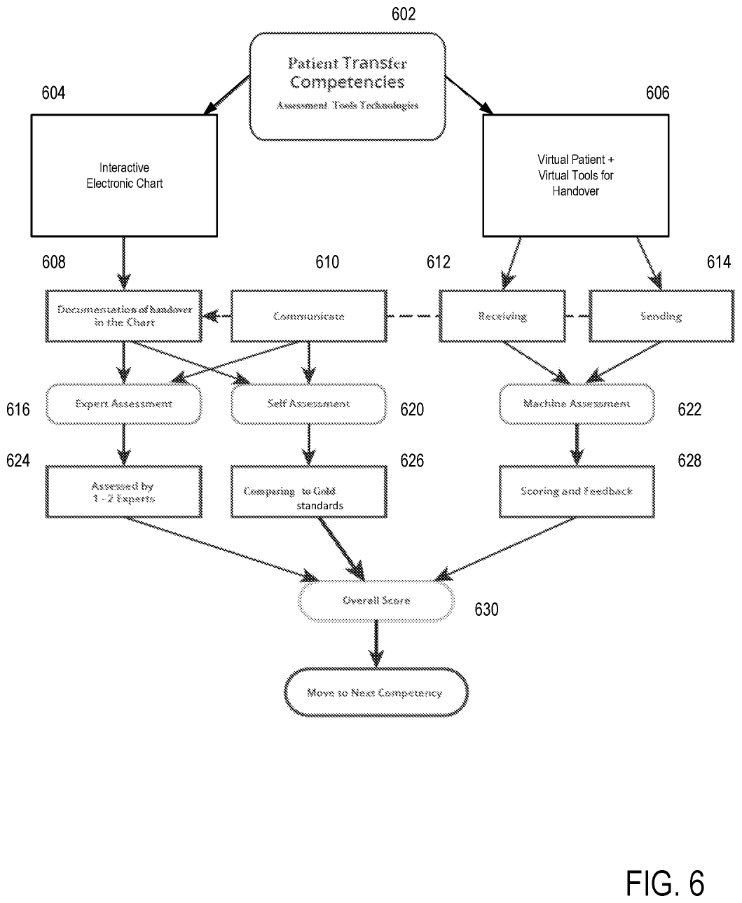

FIG. 6 shows a schematic diagram of system features for assessing patient transfer competencies in accordance with an embodiment. As shown, the system 602 comprises an interactive electronic chart 604 and a virtual patient and compensative data 606. Documentation of findings in the chart 608 and communication technologies and tools 610 facilitate an expert assessment 616 and a self-assessment to 620. The system may facilitate assessment by one or two experts to 624 and a self-assessment as compared to a gold standard 626. The system also includes special examination technologies and tools 612, and general examination technologies 614. Assessment by one or two experts 624, self-assessment by student compared to a “gold standard” 626, and scoring and feedback 628 provided by the machine assessment may all be combined into an overall score 630.

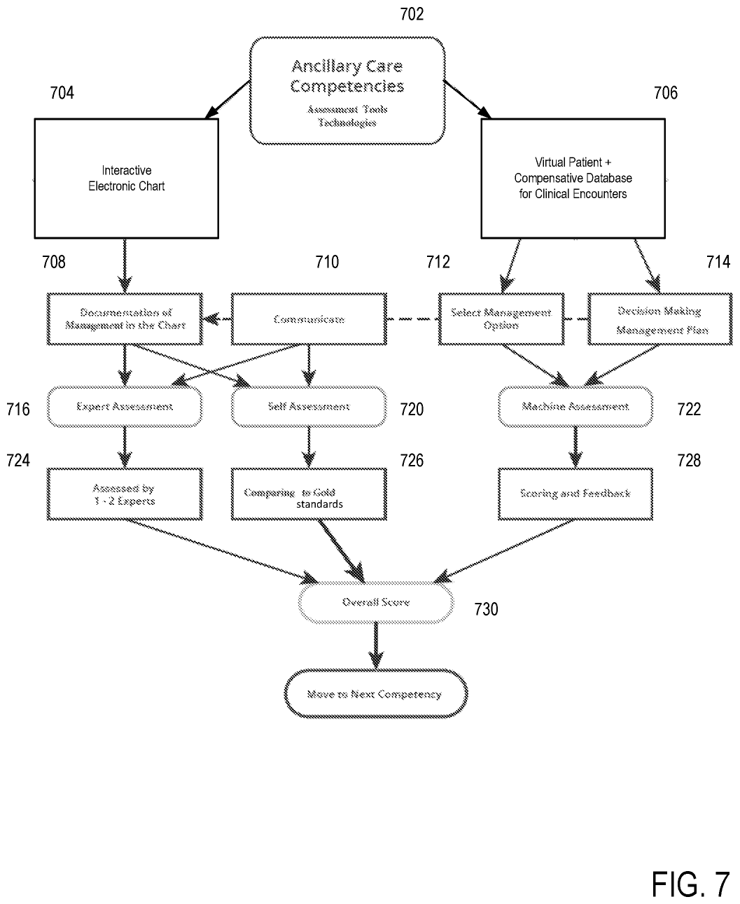

FIG. 7 shows a schematic diagram of system features for assessing ancillary care competencies in accordance with an embodiment. As shown, the system 702 comprises an interactive electronic chart 704 and a virtual patient and compensative data 706. Documentation of management in the chart 708 and communication technologies and tools 710 facilitate an expert assessment 716 and a self-assessment to 720. The system may facilitate assessment by one or two experts to 724 and a self-assessment as compared to a gold standard 726. The system also includes special examination select management options 712, and decision making management plan 714. Assessment by one or two experts 724, self-assessment by student compared to a “gold standard” 726, and scoring and feedback 728 provided by the machine assessment may all be combined into an overall score 730.

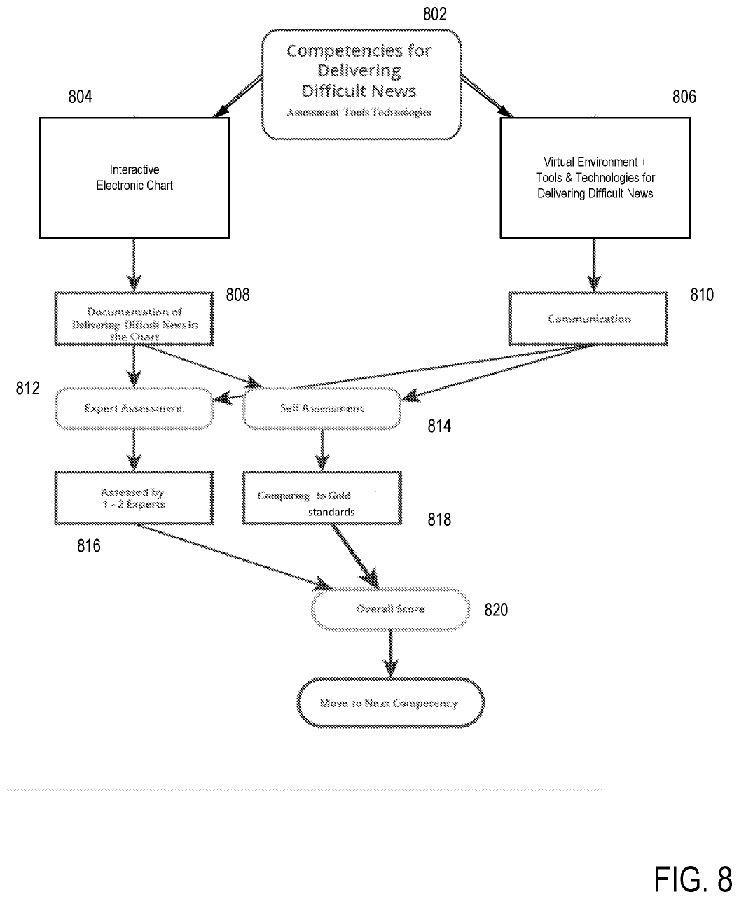

FIG. 8 shows a schematic diagram of system features for assessing competencies for delivering difficult news in accordance with an embodiment. As shown, the system 802 comprises an interactive electronic chart 804 and virtual environment tools and technologies for delivering difficult news 806. Documentation of delivering difficult news in the chart 808 and communication technologies and tools 810 facilitate an expert assessment 812 and a self-assessment to 814. The system may facilitate assessment by one or two experts to 816 and a self-assessment as compared to a gold standard 818. These may be combined into an overall score 820.

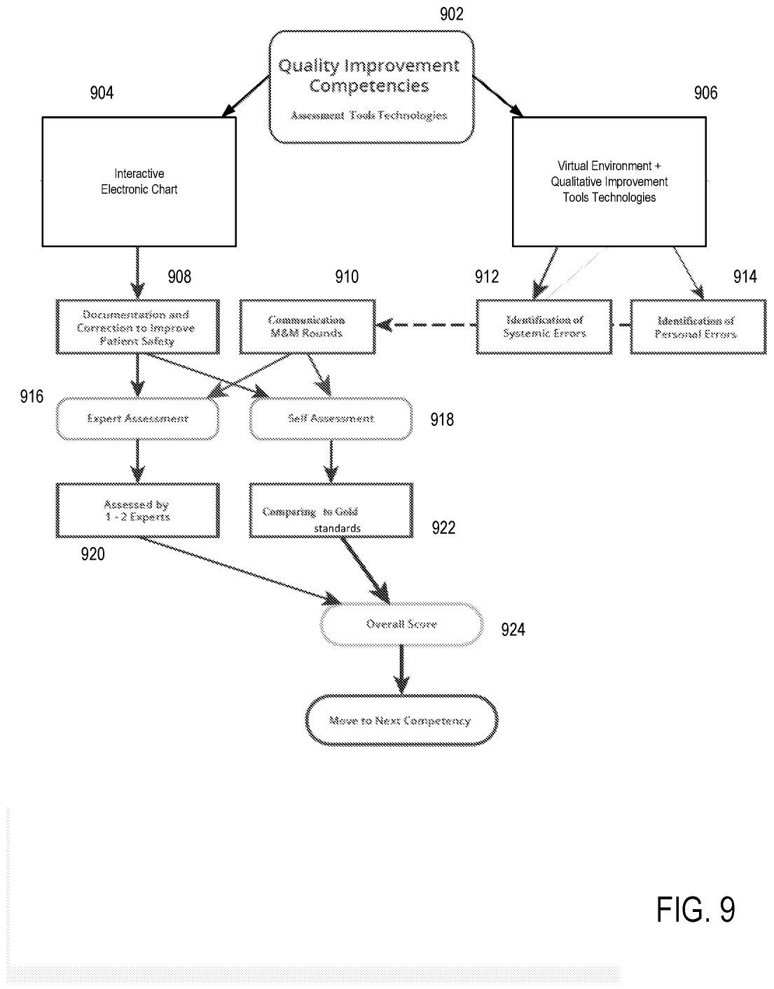

FIG. 9 shows a schematic diagram of system features for assessing quality improvement competencies in accordance with an embodiment. As shown, the system 902 comprises an interactive electronic chart 904 and virtual environment and quality improvement tools 906 for delivering difficult news. Documentation and correction to improve patient safety 908 and communication rounds 910 facilitate an expert assessment 916 and a self-assessment to 918. The system may facilitate assessment by one or two experts to 920 and a self-assessment as compared to a gold standard 922. These may be combined into an overall score 924.

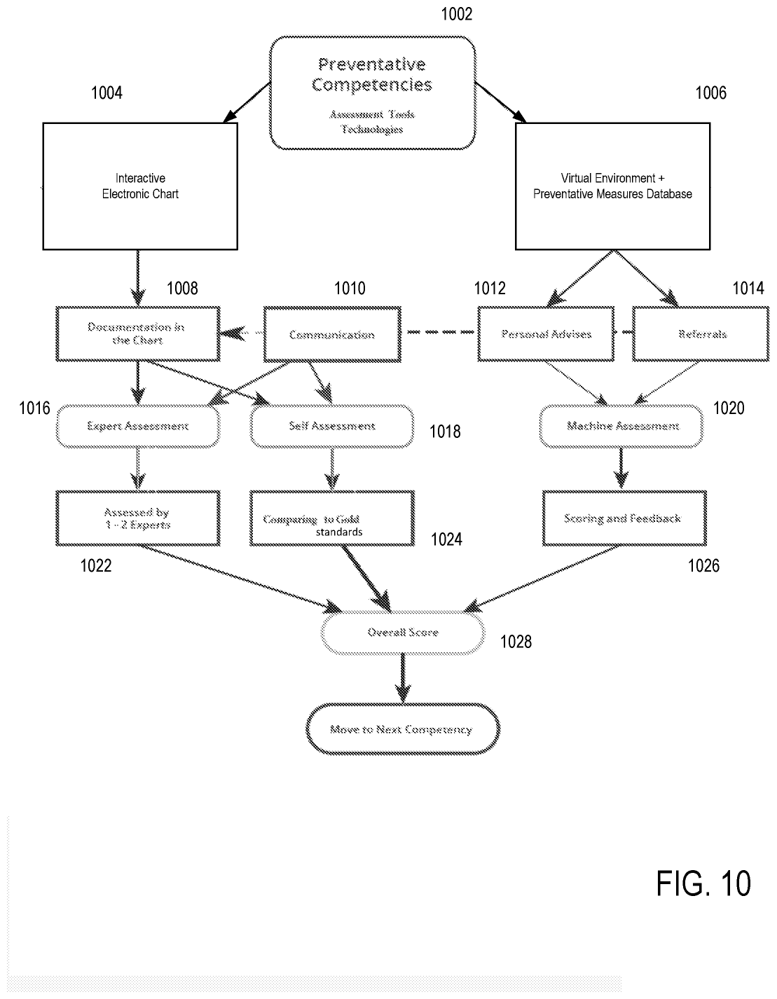

FIG. 10 shows a schematic diagram of system features for assessing preventative competencies in accordance with an embodiment. As shown, the system 1002 comprises an interactive electronic chart 1004 and a virtual environment preventative measures database 1006. Documentation of management in the chart 1008 and communication technologies and tools 1010 facilitate an expert assessment 1016 and a self-assessment to 1018. The system may facilitate assessment by one or two experts to 1022 and a self-assessment as compared to a gold standard 1024. The system also includes personal advisors 1012, and referrals 1014. Assessment by one or two experts 1022, self-assessment by student compared to a “gold standard” 1024, and scoring and feedback 1026 provided by the machine assessment may all be combined into an overall score 1028.

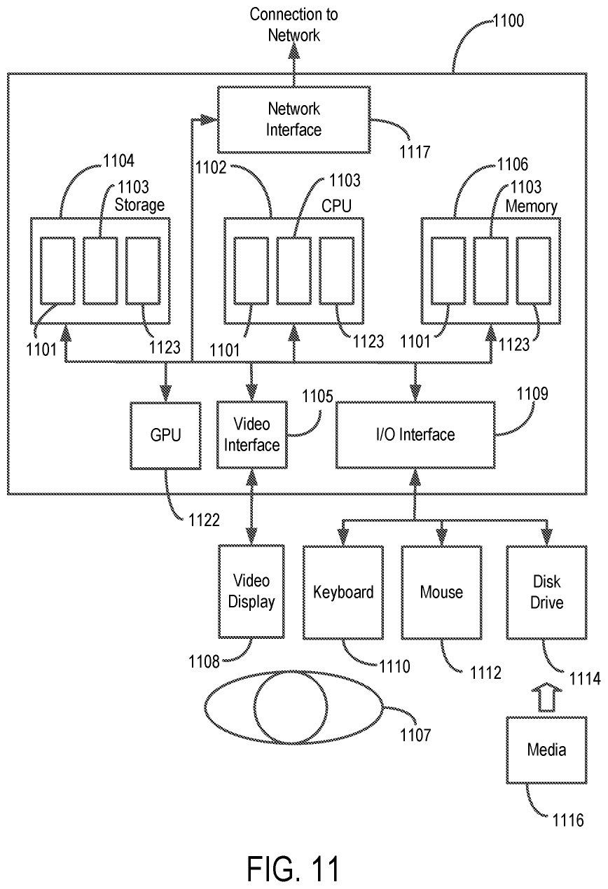

Now referring to FIG. 11 shown is a schematic block diagram of a generic computing device that may provide a suitable operating environment in one or more embodiments. A suitably configured computer device, and associated communications networks, devices, software and firmware may provide a platform for enabling one or more embodiments as described above. By way of example, FIG. 11 shows a generic computer device 1100 that may include a central processing unit (“CPU”) 502 connected to a storage unit 1104 and to a random access memory 1106. The CPU 1102 may process an operating system 1101, application program 1103, and data 1123. The operating system 1101, application program 1103, and data 1123 may be stored in storage unit 1104 and loaded into memory 1106, as may be required. Computer device 1100 may further include a graphics processing unit (GPU) 1122 which is operatively connected to CPU 1102 and to memory 1106 to offload intensive image processing calculations from CPU 1102 and run these calculations in parallel with CPU 1102. An operator 1110 may interact with the computer device 1100 using a video display 1108 connected by a video interface 1105, and various input/output devices such as a keyboard 1110, pointer 1112, and storage 1114 connected by an I/O interface 1109. In known manner, the pointer 1112 may be configured to control movement of a cursor or pointer icon in the video display 1108, and to operate various graphical user interface (GUI) controls appearing in the video display 1108. The computer device 1100 may form part of a network (see FIG. 6) via a network interface 1111, allowing the computer device 1100 to communicate with other suitably configured data processing systems or circuits. Anon-transitory medium 1116 may be used to store executable code embodying one or more embodiments of the present method on the generic computing device 1100.

Advantageously, the present system and method optimizes the assessment process in competency-based medical education by providing a virtual online platform for performing the assessment. Assessments are performed by the system's AI, by the student performing a self-assessment, and optionally by one or more subject experts who test the validity and accuracy of the machine assessment.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

Thus, in an aspect, there is provided a computer-implemented method, executable on one or more computing devices forming a part of a network, for assessment of medical competencies, comprising: providing a virtual interactive environment for access by an expert and by a student, the virtual environment including an online virtual patient; providing an artificial intelligence machine learning engine for generating random scenarios and interactions with the online virtual patient for testing competencies; and based on the student's response to the random scenarios and interactions with the online virtual patient, performing a machine assessment of the student's competency based on a machine algorithm.

In an embodiment, the method further comprises providing an expert assessment by the expert of the student's competency.

In another embodiment, the method further comprises combining the machine assessment and the expert assessment to calculate an overall score.

In another embodiment, the method further comprises comparing the machine assessment to the expert assessment to perform machine learning, and to update the machine algorithm to be used in a subsequent machine assessment.

In another embodiment, the method further comprises providing multiple expert assessments by multiple experts of the student's competency.

In another embodiment, the method further comprises assessing one or more of history taking, physical examination, investigation, management plan, patient transfer, ancillary care, delivery of difficult news, quality improvement, and preventative competencies.

In another embodiment, each of the competencies is scored and a cumulative score of all competencies is calculated.

In another embodiment, the method further comprises providing a student self-assessment of the student's competency during training sessions.

In another embodiment, the method further comprises providing the student with feedback based on the machine assessment and the assessment of one or more expert assessments.

In another embodiment, the method further comprises providing the student with recommended correction of interactions with the online virtual patient.

In another aspect, there is provided a system for performing an assessment of medical competencies, comprising: a virtual interactive environment for access by an expert and by a student, the virtual environment including an online virtual patient; and an artificial intelligence machine learning engine for generating random scenarios and interactions with the online virtual patient for testing competencies; wherein, based on the student's response to the random scenarios and interactions with the online virtual patient, the system is adapted to perform a machine assessment of the student's competency based on a machine algorithm.

In an embodiment, the system is further adapted to provide an expert assessment by the expert of the student's competency.

In another embodiment, the system is further adapted to combine the machine assessment and the expert assessment to calculate an overall score.

In another embodiment, the system is further adapted to compare the machine assessment to the expert assessment to perform machine learning, and to update the machine algorithm to be used in a subsequent machine assessment.

In another embodiment, the system is further adapted to provide multiple expert assessments by multiple experts of the student's competency.

In another embodiment, the system is further adapted to assess one or more of history taking, physical examination, investigation, management plan, patient transfer, ancillary care, delivery of difficult news, quality improvement, and preventative competencies.

In another embodiment, the system is further adapted to score each of the competencies and calculate a cumulative score of all competencies.

In another embodiment, the system is further adapted to provide a student self-assessment of the student's competency during training sessions.

In another embodiment, the system is further adapted to provide the student with feedback based on the machine assessment and the assessment of one or more expert assessments.

In another embodiment, the system is further adapted to provide the student with recommended correction of interactions with the online virtual patient.

While illustrative embodiments have been described above by way of example, it will be appreciated that various changes and modifications may be made without departing from the scope of the invention, which is defined by the following claims.

1. A computer-implemented method, executable on one or more computing devices forming a part of a network, for assessment of medical competencies, comprising:
providing a virtual interactive environment for access by an expert and by a student, the virtual environment including an online virtual patient;
providing an artificial intelligence machine learning engine for generating random scenarios and interactions with the online virtual patient for testing competencies; and
based on the student's response to the random scenarios and interactions with the online virtual patient, performing a machine assessment of the student's competency based on a machine algorithm.

2. The method of claim 1, further comprising providing an expert assessment by the expert of the student's competency.

3. The method of claim 2, further comprising combining the machine assessment and the expert assessment to calculate an overall score.

4. The method of claim 3, further comprising comparing the machine assessment to the expert assessment to perform machine learning, and to update the machine algorithm to be used in a subsequent machine assessment.

5. The method of claim 2, further comprising providing multiple expert assessments by multiple experts of the student's competency.

6. The method of claim 5, further comprising assessing one or more of history taking, physical examination, investigation, management plan, patient transfer, ancillary care, delivery of difficult news, quality improvement, and preventative competencies.

7. The method of claim 6, wherein each of the competencies is scored and a cumulative score of all competencies is calculated.

8. The method of claim 2, further comprising providing a student self-assessment of the student's competency during training sessions.

9. The method of claim 8, further comprising providing the student with feedback based on the machine assessment and the assessment of one or more expert assessments.

10. The method of claim 9, further comprising providing the student with recommended correction of interactions with the online virtual patient.

11. A system for performing an assessment of medical competencies, comprising:
a virtual interactive environment for access by an expert and by a student, the virtual environment including an online virtual patient; and
an artificial intelligence machine learning engine for generating random scenarios and interactions with the online virtual patient for testing competencies;
wherein, based on the student's response to the random scenarios and interactions with the online virtual patient, the system is adapted to perform a machine assessment of the student's competency based on a machine algorithm.

12. The system of claim 11, wherein the system is further adapted to provide an expert assessment by the expert of the student's competency.

13. The system of claim 12, wherein the system is further adapted to combine the machine assessment and the expert assessment to calculate an overall score.

14. The system of claim 13, wherein the system is further adapted to compare the machine assessment to the expert assessment to perform machine learning, and to update the machine algorithm to be used in a subsequent machine assessment.

15. The system of claim 12, wherein the system is further adapted to provide multiple expert assessments by multiple experts of the student's competency.

16. The system of claim 15, wherein the system is further adapted to assess one or more of history taking, physical examination, investigation, management plan, patient transfer, ancillary care, delivery of difficult news, quality improvement, and preventative competencies.

17. The system of claim 16, wherein the system is further adapted to score each of the competencies and calculate a cumulative score of all competencies.

18. The system of claim 12, wherein the system is further adapted to provide a student self-assessment of the student's competency during training sessions.

19. The system of claim 18, wherein the system is further adapted to provide the student with feedback based on the machine assessment and the assessment of one or more expert assessments.

20. The system of claim 19, wherein the system is further adapted to provide the student with recommended correction of interactions with the online virtual patient.
